(19) United States
(12) Patent Application Publication
(10) Pub. No.: US 2014/0004600 A1
(43) Pub. Date: Jan. 2, 2014

(54) METHODS AND APPARATUS FOR CULTIVATING PHOTOAUTOTROPHIC ORGANISMS

(71) Applicants: Vadim Tarassov, West New York, NJ (US); Ennio Verderese, Stoneham, MA (US); Joseph McCarthy, Lowell, MA (US)

(72) Inventors: Vadim Tarassov, West New York, NJ (US); Ennio Verderese, Stoneham, MA (US); Joseph McCarthy, Lowell, MA (US)

(73) Assignee: FUEL CELL EARTH LLC, Wakefield, MA (US)

(21) Appl. No.: 13/720,979

(22) Filed: Dec. 19, 2012

Related U.S. Application Data
(60) Provisional application No. 61/577,316, filed on Dec. 19, 2011.

(51) Int. Cl. C12M 1/00

(52) U.S. Cl. CPC C12M 21/02
USPC 435/292.1; 156/253

(57) ABSTRACT

A replaceable photobioreactor for use within a cultivation system for growing photosynthetic organisms in a liquid growth medium is disclosed. The replaceable photobioreactor is used holding a liquid growth medium while exposing the photosynthetic organisms to light. A ‘dark tank’ is used in addition to the replaceable photobioreactor for holding the liquid growth medium in darkness such that a day and night cycle may be simulated.

### RELATED APPLICATIONS

The present patent application claims the benefit of the previous U.S. Provisional Patent Application entitled “Methods and Apparatus for Cultivating Photoautotrophic Organisms” filed on Dec. 19, 2011 having Ser. No. 61/557,891.

### TECHNICAL FIELD

The present invention relates to the field of cultivating photoautotrophic organisms. In particular, but not by way of limitation, the present disclosure teaches techniques for the cultivation of the photoautotrophic organisms such as microalgae.

### BACKGROUND

The price of oil has sharply increased over the past decade and is projected to increase further as oil demand increases and existing oil fields deplete. To help supplement the petroleum supply, energy producers are increasingly turning to biofuels such ethanol and biodiesel that create liquid fuels from biomass.

One promising growth area for biofuels is oil produced by algae. Many algae species have evolved the ability to synthesize oil. Algae evolved the ability to synthesize oil since oil is less dense than water such that the algae that synthesized oil floated to the water surface where sunlight can be obtained for growth.

In addition to oil, other biomass products may be produced from cultivated algae. For example, nutritional supplements and pharmaceuticals may be manufactured from algae biomass harvest after growing a crop of algae. Due to all of the useful products that may be created from algae, it would be desirable to improve the efficiency of the techniques and the apparatus used for cultivating algae.

### BRIEF DESCRIPTION OF THE DRAWINGS

In the drawings, which are not necessarily drawn to scale, like numerals describe substantially similar components throughout the several views. Like numerals having different letter suffixes represent different instances of substantially similar components. The drawings illustrate generally, by way of example, but not by way of limitation, various embodiments discussed in the present document.

FIG. 1 illustrates a computer system within which a set of instructions, for causing the machine to perform any one or more of the methodologies discussed herein, may be executed.

FIG. 2 illustrates a process diagram of a complete photosynthetic organism cultivation system comprising a small starter cultivation system and a larger production cultivation system.

FIG. 3 illustrates an example of a cross flow algae cultivation system in one embodiment.

FIG. 4A illustrates a front view of a replaceable transparent cross flow photobioreactor.

FIG. 4B illustrates a side view of a replaceable transparent cross flow photobioreactor.

FIG. 5 illustrates some examples of the possible cross-section shapes of photobioreactor channels.

FIG. 6A illustrates a cross-section of single photobioreactor that is surrounded on both sides by two artificial lighting panels.

FIG. 6B illustrates a cross-section of a single photobioreactor with an artificial lighting panel on one side and a reflective surface on the other side.

FIG. 6C illustrates a cross-section of a system with two side-by-side photobioreactors wherein an artificial lighting panel is placed on each side.

FIG. 6D illustrates a photobioreactor with a transparent sheet on the side close to a lighting panel and a light reflecting sheet on the other side.

FIG. 7A illustrates a cross-section view of an enclosed artificial raceway pond embodiment.

FIG. 7B illustrates an over-head view of an artificial raceway pond embodiment with light panels positioned over the raceway pond.

FIG. 7C illustrates an over-head view of an artificial raceway pond embodiment with light panels retracted away from the raceway pond.

FIG. 8A illustrates an isometric view of an artificial raceway pond embodiment.

FIG. 8B illustrates an isometric view of an artificial raceway pond made up with three modulator raceway pond units.

FIG. 8C illustrates an over-head view of an artificial raceway pond made up with five modulator raceway pond units with light panels positioned over the raceway pond units.

FIG. 8D illustrates an over-head view of an artificial raceway pond made up with five modulator raceway pond units with light panels retracted away from the raceway pond units.

FIG. 9A illustrates a cultivation system with a raceway pond having dark tank pipes on both sides.

FIG. 9B illustrates a cultivation system with a dark tank pipe on only one side.

FIG. 10A illustrates a cultivation system with dark tank pipes horizontally adjacent to raceway pond units.

FIG. 10B illustrates a cultivation system with dark tank pipes vertically below raceway pond units.

FIG. 11 illustrates three cultivation systems stacked vertically on top of each other.

FIG. 12 illustrates a first example of a harvesting system for an algae cultivation system.

FIG. 13 illustrates a second example of a harvesting system for an algae cultivation system that is designed to collect oil.

### DETAILED DESCRIPTION

The following detailed description includes references to the accompanying drawings, which form a part of the detailed description. The drawings show illustrations in accordance with example embodiments. These embodiments, which are also referred to herein as “examples,” are described in enough detail to enable those skilled in the art to practice the invention. It will be apparent to one skilled in the art that specific details in the example embodiments are not required in order to practice the present invention. For example, although some of the example embodiments are disclosed with reference to cultivating algae, many of the disclosed teachings can be used in many other environments. The example embodiments may be combined, other embodiments may be utilized, or structural, logical and electrical changes may be made without departing from the scope of what is claimed. The following detailed description is, therefore, not to be taken in a limiting sense, and the scope is defined by the appended claims and their equivalents.

In this document, the terms “a” or “an” are used, as is common in patent documents, to include one or more than one. In this document, the term “or” is used to refer to a nonexclusive or, such that “A or B” includes “A but not B,” “B but not A,” and “A and B,” unless otherwise indicated. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

Computer Systems

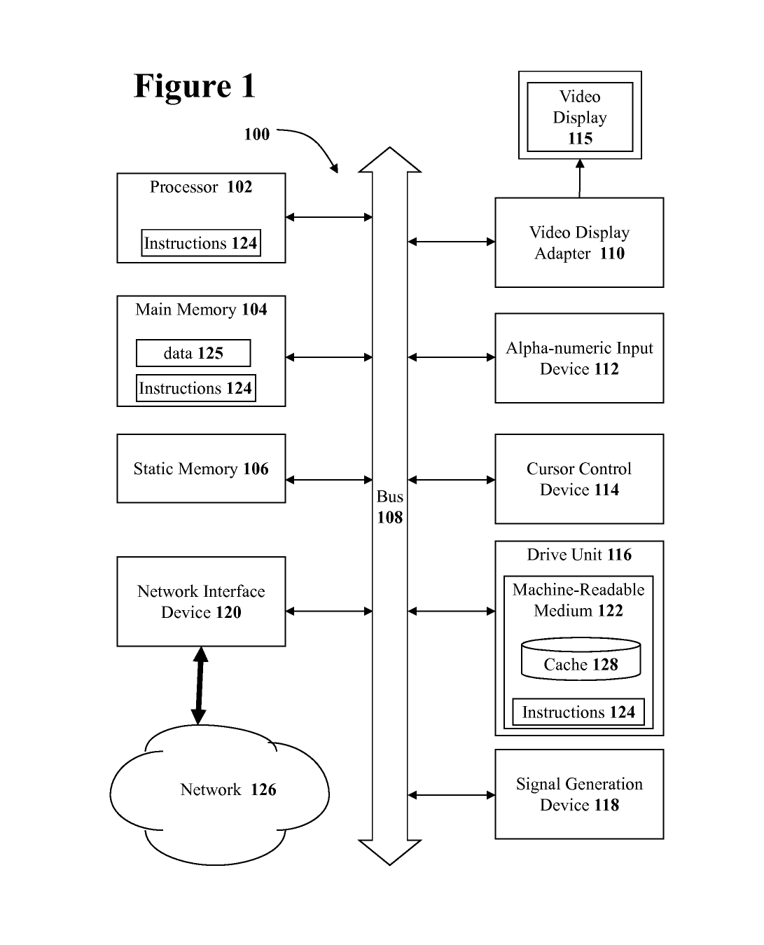

The present disclosure concerns techniques and apparatus for cultivating algae. To monitor and control the cultivation process, computer systems may be used. FIG. 1 illustrates a diagrammatic representation of a machine in the example form of a computer system 100 that may be used to implement portions of the present disclosure. Within computer system 100 of FIG. 1, there are a set of instructions 124 that may be executed for causing the machine to perform any one or more of the methodologies discussed within this document. Furthermore, while only a single computer is illustrated, the term “computer” shall also be taken to include any collection of machines that individually or jointly execute a set (or multiple sets) of instructions to perform any one or more of the methodologies discussed herein.

The example computer system 100 of FIG. 1 includes a processor 102 (e.g., a central processing unit (CPU), a graphics processing unit (GPU) or both) and a main memory 104 and a static memory 106, which communicate with each other via a bus 108. The computer system 100 may further include a video display adapter 110 that drives a video display system 115 such as a Liquid Crystal Display (LCD). The computer system 100 also includes an alphanumeric input device 112 (e.g., a keyboard), a cursor control device 114 (e.g., a mouse or trackball), a disk drive unit 116, a signal generation device 118 (e.g., a speaker) and a network interface device 120. Note that not all of these parts illustrated in FIG. 1 will be present in all embodiments. For example, a computer server system may not have a video display adapter 110 or video display system 115 if that server is controlled through the network interface device 120.

The disk drive unit 116 includes a machine-readable medium 122 on which is stored one or more sets of computer instructions and data structures (e.g., instructions 124 also known as ‘software’) embodying or utilized by any one or more of the methodologies or functions described herein. The instructions 124 may also reside, completely or at least partially, within the main memory 104 and/or within a cache memory 103 associated with the processor 102. The main memory 104 and the cache memory 103 associated with the processor 102 also constitute machine-readable media.

The instructions 124 may further be transmitted or received over a computer network 126 via the network interface device 120. Such transmissions may occur utilizing any one of a number of well-known transfer protocols such as the well-known File Transport Protocol (FTP). While the machine-readable medium 122 is shown in an example embodiment to be a single medium, the term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that cause the machine to perform any one or more of the methodologies described herein, or that is capable of storing, encoding or carrying data structures utilized by or associated with such a set of instructions. The term “machine-readable medium” shall accordingly be taken to include, but not be limited to, solid-state memories, optical media, and magnetic media.

For the purposes of this specification, the term “module” includes an identifiable portion of code, computational or executable instructions, data, or computational object to achieve a particular function, operation, processing, or procedure. A module need not be implemented in software; a module may be implemented in software, hardware/circuitry, or a combination of software and hardware.

Cultivation System Overview

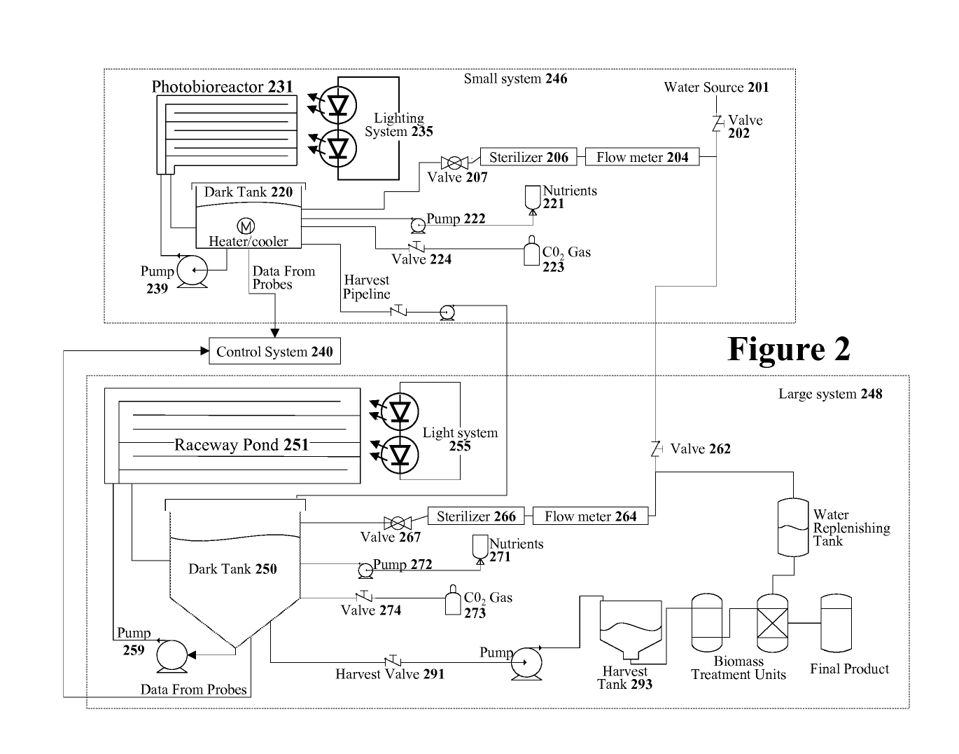

FIG. 2 illustrates a process diagram of one embodiment of a photosynthetic organism cultivation system. The photosynthetic organism cultivation system of FIG. 2 actually includes both a small starter cultivation system 246 and a larger production cultivation system 248. However, a photosynthetic organism cultivation system may be constructed with only the small starter cultivation system 246 or only the large production cultivation system 248.

The photosynthetic cultivation system of FIG. 2 is modular and comprises fully automated combined closed-type photobioreactor. The photosynthetic organism cultivation system fully meets the requirements of mixotrophic or heterotrophic growth of the photosynthetic organisms and sterility of the culture. The photosynthetic organism cultivation system may be used to cultivate algae or any other photosynthetic organism that can be grown in a liquid growth medium.

The small starter cultivation system 246 can serve as a photoautotrophic organism inoculator for any kind of larger industrial cultivation photobioreactors (open or closed systems, like shallow or stirred ponds, tank reactors, etc.). For example, FIG. 2 illustrates small starter cultivation system 246 acting as an inoculator for larger production cultivation system 248. Alternatively, the small starter cultivation system 246 may serve as a small-scale independent unit for photoautotrophic organism cultivation and research. In a research capacity, it is important to have sterile equipment and the ability to accurately reproduce experimental runs under the same conditions.

The small starter cultivation system 246 accepts water from a water source 201 that passes through valve 202, flow meter 204, sterilizer 206, and valve 207 into a dark tank 220. The water serves as the liquid base for a liquid growth medium for photosynthetic organisms. The growth medium dark tank 220 is seeded with the source photosynthetic organisms (such as algae) to be cultivated. The photosynthetic organisms in the liquid growth medium are pumped (with pump 239) through a photobioreactor 231 where the photosynthetic organisms are exposed to a lighting system. The lighting system may comprise access to natural sunlight or artificial light system 235 as illustrated in FIG. 2. In one embodiment, an energy efficient Light Emitting Diode (LED) based lighting system is used.

The liquid growth medium in the dark tank 220 is supplemented with nutrients from nutrients source 221 such as fertilizers through controlled pump system 222. The liquid growth medium in the dark tank 220 is also supplemented with CO₂ gas from CO₂ gas source 223 through controlled valve 224. The light from light system 235, the nutrients from nutrients source 221, and the CO₂ gas from CO₂ gas source 223 provide the photosynthetic organisms within the cultivation system 246 with necessary ingredients to reproduce and grow.

A collection of sensor probes monitor several different parameters of the liquid growth medium. In the small system 246 embodiment of FIG. 2, the data probes are coupled to the dark tank 220. However, in the same system or in alternative embodiments the sensor probes may be located in other parts of the cultivation system 246. The sensor probes may include but are not limited to temperature probes, pH (acidity) probes, electrical conductivity probes, dissolved oxygen (OD) probes, and oxidation-reduction potential (ORP) probes. The sensor probes are coupled to a control system 240 that receives processes the data from the sensor probes. The control system 240 may record all of the data collected for later analysis.

In a preferred embodiment, some or all of the data from the control system 240 is made available on a secure internet coupled computer system. In this manner, the data from the sensor probes can be accessed at any time and changes may be made remotely to the control system 240 by an operator with access to the secure internet coupled computer system. The control system 240 can also be programmed to send out alerts via email, text messages, or any other digital communication system when the cultivation system determines that user attention is required to handle an issue. For example, a technician may be alerted if the cultivation system is unable to keep the various cultivation system parameters (such as temperature, oxygen level, pH level, etc.) within a defined set of ranges.

The control system 240 may react to the data received on the sensor probes by changing composition and state of the liquid growth medium in various different manners. The control system 240 may control valve 207 when more water is needed to dilute the liquid growth medium. The control system 240 may control pump 222 when more nutrients from nutrients source 221 are needed in the liquid growth medium. The control system 240 may control valve 224 when more CO₂ gas from CO₂ gas source 223 is needed in the liquid growth medium for the photosynthetic organisms. The control system 240 may adjust the heater/cooler in the dark tank 220 to keep the liquid growth medium within a desired temperature range. The control system 240 may adjust the parameters of the lighting system 235 to adjust the light intensity or light schedule as desired. The control system 240 may control the pump 239 used to circulate the growth medium between the photobioreactor 231 and dark tank 220 to create a desired day/night rhythm for the photosynthetic organisms. When a batch of the photosynthetic organisms has completed a growth cycle, the control system 240 may open a valve on a harvest pipeline to transfer the photosynthetic organisms from the small scale system 246 to the large scale production system 248 or to other required procedures.

The large scale production system 248 may operate in similar manner to the small scale system 246 except that the large scale production system 248 will generally have a combination of two photobioreactors: cross-flow photobioreactor similar to the small scale system 246 but with much larger components and raceway pond system followed by an output system for harvesting a final product. The large scale production system 248 uses a larger raceway pond system 251 that serves as the photobioreactor for the photosynthetic organisms. The light system 255 for the larger raceway pond system 251 may be access to natural sunlight or a larger array of artificial light sources such as LEDs. The large scale production system 248 also includes a much larger dark tank 250. The dark tank 250 is coupled to similarly up-scaled system components such as the water system providing sterilized water through valve 267, growth medium nutrients source 271 through pump 272, and CO₂ gas source 273 through valve 271.

Like the small scale system 246, the large scale production cultivation system 248 is controlled by a control system. In the embodiment of FIG. 2, the same control system 240 is used to control both the small scale system 246 and the large scale production system 248. However, in other embodiments the two different cultivation systems may use separate control systems. The control system 240 may monitor the same types of liquid growth medium parameters (temperature, pH, electrical conductivity, dissolved oxygen (OD), oxidation reduction potential (ORP), etc.) in the large scale production system 248 as in the small scale cultivation system 246. The control system 240 may also adjust the liquid growth medium of the large scale production system 248 by controlling water valve 267, nutrients pump 272, CO₂ gas valve 274, photobioreactor pump 259, light system 255, harvest valve 291, and other controllable elements.

When the large scale production system 248 has completed a cultivation of photosynthetic organisms, the control system 240 may open harvest valve 291 to harvest the crop of photosynthetic organisms by moving the photosynthetic organisms into harvest tank 293. The photosynthetic organisms in harvest tank 293 are then processed by harvesting systems that will be described in greater detail in a later section of this document.

Cultivation System Details

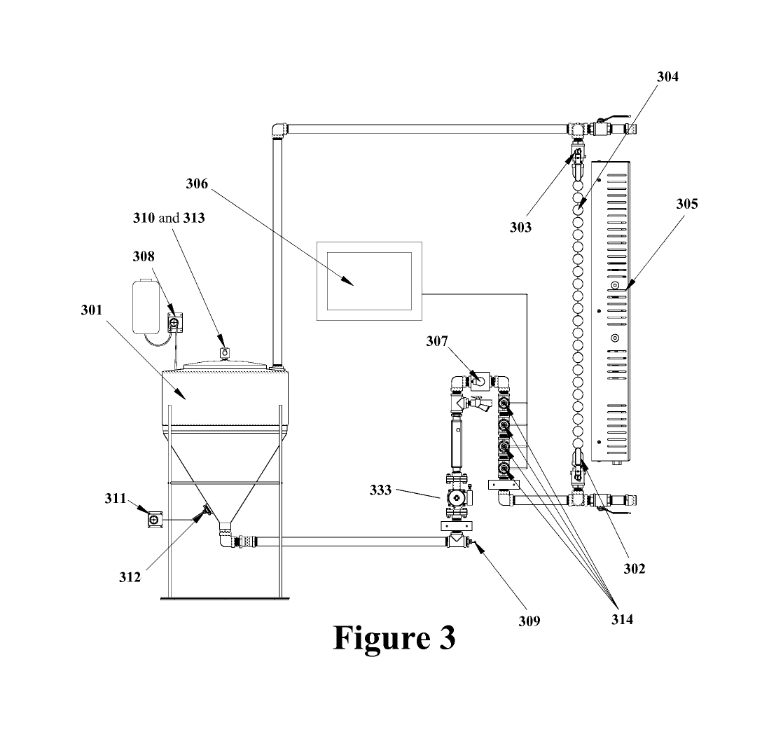

FIG. 3 illustrates an example of a cross flow cultivation system in one embodiment. The cross flow cultivation system consists of the non-transparent dark tank 301 that contains the liquid growth medium (water, nutrients, CO₂ gas, etc.) that hosts the photoautotrophic organisms. The dark tank 301 is coupled through a pump 333 to a lower (inlet) manifold 302. The pump 333 may be controlled by a control system 306 that controls the overall operation of the cultivation system. The lower (inlet) manifold 302 couples to the bottom of a removable transparent cross flow photobioreactor 304. FIG. 3 illustrates the removable transparent cross flow photobioreactor 304 in cross-section form. Additional details about the removable transparent cross flow photobioreactor 304 will be presented in the next section.

The cultivation system pumps liquid growth medium with the photosynthetic organisms from the dark tank 301 through the removable transparent cross flow photobioreactor 304, out of an upper (outlet) manifold 303, and back to the dark tank 301 where the liquid growth medium containing the photosynthetic organisms is collected. When passing through the removable transparent cross flow photobioreactor 304, the liquid growth medium with the photosynthetic organisms is exposed to light from an artificial lighting system 305 or to natural sunlight (not shown). The liquid growth medium with the photosynthetic organisms passes through photobioreactor 304 and returns to directed to the dark tank 301 thus completing the circulation cycle.

Note that many cross flow photobioreactors may be coupled in parallel to each other (not shown). Thus, the size of cultivation system may be adjusted by determining how many cross flow photobioreactors will be used. An installed cultivation system may be increased in size by adding additional cross flow photobioreactors.

In the embodiment of FIG. 3, a set of sensor probes 314 are coupled to the piping of the cultivation system. The set of sensor probes 314 may include sensors for measuring pH (acidity), temperature, electrical conductivity, dissolved oxygen (OD), oxidation reduction potential (ORP), and other important parameters of the liquid growth medium. In the embodiment of FIG. 3, the set of sensor probes 314 are assembled in one unit, which is placed near the inlet of the removable transparent cross flow photobioreactor 304. The sensor probes 314 are all coupled to the control system 306 that may record the data measurements from the sensor probes 314. The control system 306 reacts in response to the data from the sensor probes 314 to control the state of the liquid growth medium.

The automated control system 306 monitors and controls the cultivation process. The control system 306 may comprise a computer system as disclosed in FIG. 1. As set forth in the previous paragraph, the control system 306 is supported by a set of sensor probes 314 that acquire data about the liquid growth medium. Using data from the sensor probes 314 and a user-defined cultivation protocol, the control system 306 controls solenoids; automatic valves 307; nutrients dosing device 308; CO₂ dosing device 309; an automatic valve for oxygen and excessive air retrieval 310; an air pump 311; an air diffuser 312; a vacuum pump 313, and any other controllable elements of the cultivation system.

The control system 306 may be programmed to keep the measured liquid growth medium parameters within a set of defined limits. The control system may operate on cyclical schedule designed to cause the photosynthetic organisms to grow and reproduce as fast as possible. The control system 306 also monitors the harvesting process by periodically dosing the quantity of liquid growth medium with the photoautotrophic organisms harvested from the tubular photobioreactor and the process of equal replenishment of the photobioreactor by the liquid medium (typically water) from the external or recirculating source.

The dark tank 301 is where much of the monitoring and adjusting of the liquid growth medium is handled. For example, the sterilized nutrients dosing device 308 may add nutrients into the liquid growth medium under the control of control system 306. Similarly, an air pump 311 pumps sterilized air to an air diffuser 312 coupled to dark tank 301 to ensure that the photoautotrophic organism's liquid growth medium receives aeration to prevent oxygen super-saturation. Another option is to use a vacuum pump 313 connected to the top of the recirculation tank to facilitate oxygen removal from the liquid culture medium. An automatic valve 310 mounted on the top of the dark tank 301 may be used for oxygen and excessive gases retrieval.

In the embodiment of FIG. 3, a CO₂ diffuser and CO₂ dosing device 309 are coupled to the piping of the cultivation system to add CO₂ gas from an external CO₂ tank (not shown). The CO₂ gas is used by the photosynthetic organisms to grow and reproduce. The CO₂ diffuser and CO₂ dosing device 309 may use hollow fiber membranes that effectively disperse CO₂ into the growth medium. For addition dispersion and to avoid CO₂ volatilization in the dark tank 301 the CO₂ diffuser is placed proximate to the inlet of a recirculation pump 333 such that the recirculation pump 333 helps mix the CO₂ gas into the liquid growth medium.

The lighting system 305 may comprise flat panels covered with evenly distributed Light Emitting Diodes (LEDs) that emit light in the Photosynthetically Active Radiation (PAR) range of spectrum. The PAR range is generally in the 400 nanometer to 700 nanometer region of the electromagnetic spectrum. In one embodiment, the flat panels of the lighting system 305 comprise a substantially evenly distributed array of red and blue LEDs. The surface area of a lighting panel is substantially equal in size to the surface area of photobioreactor 304 thereby ensuring the even distribution of light intensity across the surface of photobioreactor 304.

Photobioreactor Details

In one embodiment, the photobioreactor 304 of the cultivation system is a removable transparent cross flow system. Such a removable photobioreactor 304 allows for easy replacement if the photobioreactor 304 has become less transparent due to residue accumulating on the inside surface of the photobioreactor 304. A removable photobioreactor 304 also helps ensures that the cultivation system will be sterile between cultivating batches of different strains of photosynthetic organisms.

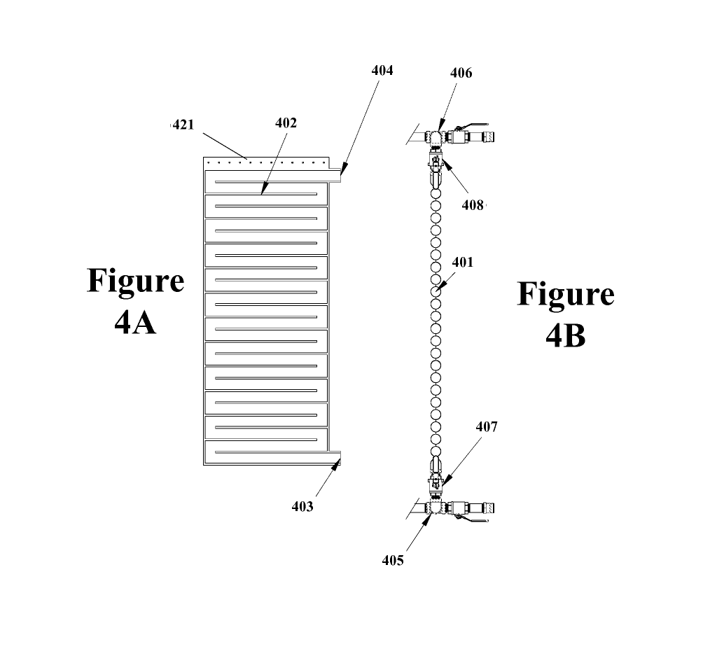

FIGS. 4A and 4B illustrate front and side views of a removable transparent cross flow photobioreactor 401, respectively. Referring to FIG. 4A, the removable transparent cross flow photobioreactor 401 may be constructed with two plastic sheets having good light transmission properties. The two plastic sheets may be welded together using a heat process or chemical adhesives. The two transparent plastic sheets may be welded in a way to form a continuous coil-pipe type channel 402 in between the plastic sheets. The inlet and outlet of the continuous coil-pipe channel are ended with connecting pipes 403 and 404. Connecting pipes 403 and 404 of the photobioreactor 401 are formed such that they may be easily coupled to 3-way valves 407 and 408 of a cultivation system. The top of the removable transparent cross flow photobioreactor 401 has a hanging system connector 421 that allows the photobioreactor 401 to be hung up using a number of small holes in the hanging system connector 421.

Referring to FIG. 4B, connections to inlet 405 and outlet 406 manifolds are realized through 3-way valves 407 and 408 for emptying a photobioreactor 401 in case of replacement without interrupting cultivation process in other photobioreactor 401 in the same cultivation system. The transparent cross flow photobioreactor 401 may be sterilized during manufacture to avoid the need for any complicated sterilization process when the photobioreactors 401 are already mounted. The total photobioreactor capacity may be increased by adding number of the photobioreactors in parallel between manifolds 405 and 406.

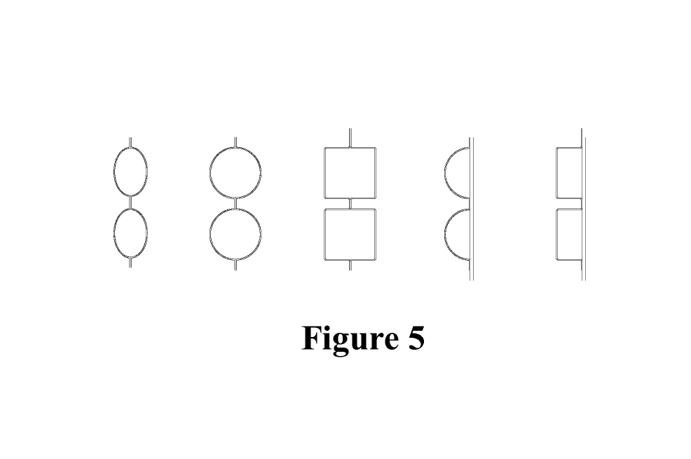

The cross-flow channels of the photobioreactors may have different shapes in cross-section. Some examples of the possible cross-section shapes of photobioreactor channels are illustrated in FIG. 5. Plastic sheets may be welded, molded, or otherwise manufactured to form the continuous cross flow channel. The cross flow channel may have other shapes than the examples illustrated in FIG. 5.

The two plastic sheets used to form a replaceable photobioreactor may both be transparent for use in a combination of natural and artificial lighting environment. Specifically, natural light may be provided to one side of the replaceable photobioreactor during the day and artificial lighting maybe exposed to the other side of the replaceable photobioreactor during night.

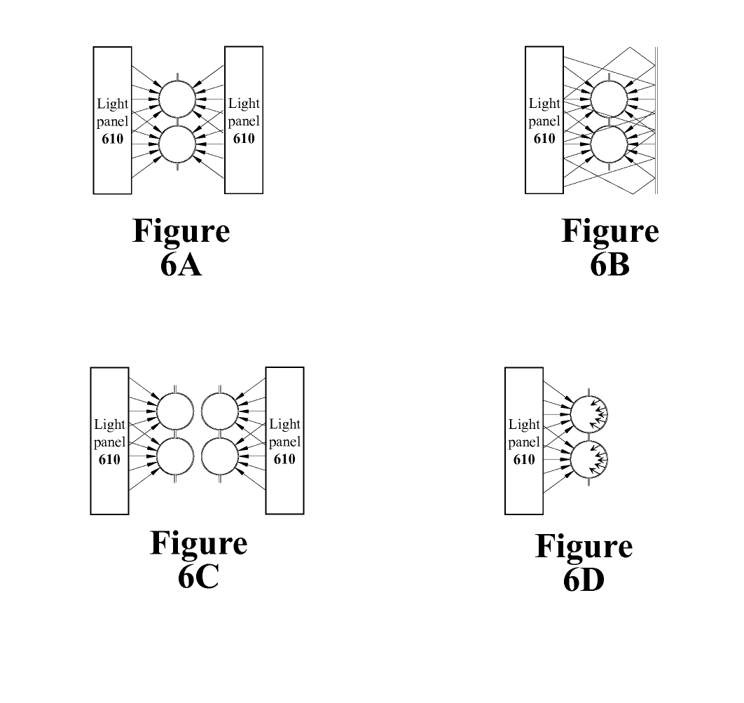

Artificial lighting may be used with the photobioreactor in various different manners. FIGS. 6A, 6B, 6C, and 6D illustrate example cross-section views of four different systems of using photobioreactors with artificial lighting. (Note that natural lighting may be used instead of the artificial lighting illustrated in FIGS. 6A, 6B, 6C, and 6D.) FIG. 6A illustrates a subsection of a single photobioreactor that is surrounded on both sides by two artificial lighting panels 610. In this manner, the single photobioreactor is saturated with artificial light from both sides. FIG. 6B illustrates a subsection of a single photobioreactor with an artificial lighting panel 610 on one side and a reflective surface on the other side. In the arrangement of FIG. 6B the light that does not directly strike the photobioreactor or passes through the photobioreactor may be reflected back and strike the photobioreactor on a second pass. FIG. 6C illustrates a system with two side-by-side photobioreactors wherein an artificial lighting panel 610 is placed on each side. In the arrangement of FIG. 6C the light that does not directly strike the nearby photobioreactor or passes through the nearby photobioreactor may be absorbed by the other photobioreactor.

In some embodiments, the photobioreactor may be constructed with one transparent sheet and one light-reflecting sheet to fully use the light flux directed toward the single transparent side of photobioreactor. FIG. 6D illustrates a photobioreactor with a transparent sheet on the side close to the lighting panel 610 and a light-reflecting sheet on the other side. The light that passes through the growth medium and strikes the light-reflecting sheet will bounce back and have a second chance of striking a photosynthetic organism in the liquid growth medium. The two right-most cross sections illustrated in FIG. 5 may be well suited for using a light-reflecting sheet on the right side of the photobioreactor.

To improve light distribution inside of photobioreactor channels, a limited quantity of shiny plastic beads may be placed into the growth medium. The density of plastic material may be substantially similar to water density such that the material easily travels in the growth medium. The hardness of the plastic material should be less than the hardness of the material that is used to form photobioreactor to avoid scratching the photobioreactor walls and thereby reducing light transmission into the photobioreactor. The diameter of plastic beads will generally be substantially larger than the dimension of the cultivated photosynthetic organisms in order to avoid the damage of photosynthetic organisms in case of contact. The plastic beads may also serve other useful purposes within the liquid growth medium. For example, the plastic beads help mix the growth medium and serve as a photobioreactor cleaning tool that helps keep the photobioreactor clean by brushing against the sides of the photobioreactor and thereby removing accumulated material.

Modular Raceway Pond System

As set forth in the system diagram of FIG. 2 the teachings of the present disclosure may be used to construct a small starter cultivation system 246 and/or a larger production cultivation system 248. A traditional outdoor pond may be used in some embodiments of a large production cultivation system 248. However, an enclosed artificial raceway pond system provides many advantages that are not available with a traditional outdoor pond.

An enclosed artificial pond system prevents diseases and outside contaminants (such as heavy metals or other toxins) from harming the photosynthetic organisms. Furthermore, an enclosed artificial pond system reduces evaporation of the liquid growth medium. Having a closed system prevents the loss of nutrients and gases injected into the liquid growth medium. With a closed system, it is much easier to maintain a consistent environment for photosynthetic organism growth. Specifically, it is much easier to keep temperature, dissolved gas mixture, and nutrient parameters of the liquid growth medium within a defined range that has been selected for a growth cycle.

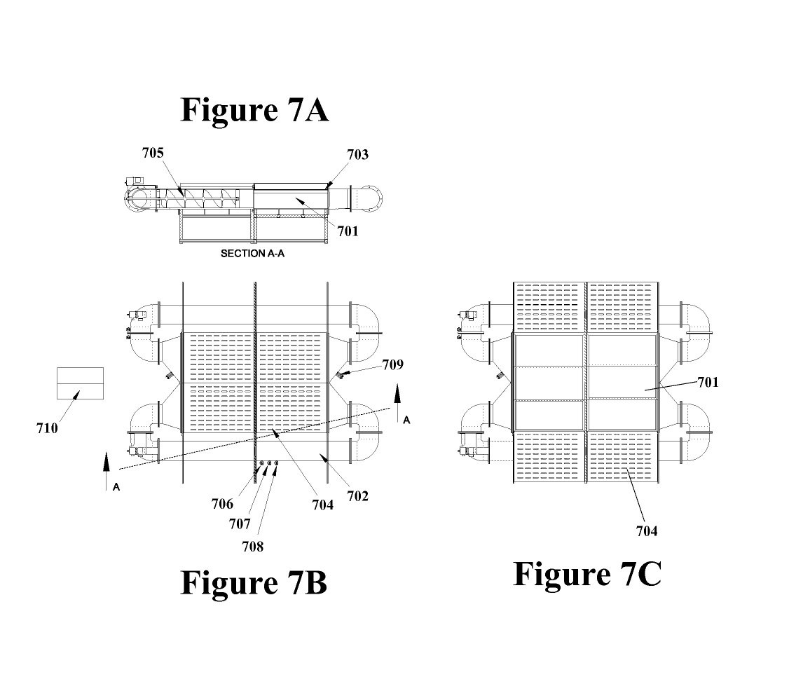

FIGS. 7A, 7B, and 7C illustrate an example of an enclosed artificial raceway pond. FIG. 7A illustrates a cross-section view of the enclosed artificial raceway pond along line A-A of the top down view of FIG. 7B. The cross-section view illustrates a main pool region 701 that is covered with transparent material 703. By filling the raceway pond all the way up with liquid growth medium such that the liquid growth medium contacts the transparent material 703, the enclosed system prevents having an air layer above the liquid growth medium. This prevents condensation from forming that will reduce the transmission of light into the raceway pond. Furthermore, contaminants in the air may harm the liquid growth medium.

The transparent material 703 may be covered with flat LED light distributing panels 704 as illustrated in FIG. 7B. The flat LED light distributing panels 704 may be constructed with evenly distributed LEDs emitting light in the RAR range of spectrum. The flat LED light distributing panels 704 may be retracted to allow for natural lighting to be used as illustrated in FIG. 7C.

The main pool region 701 is supplemented with a “dark” tank formed by pipes 702. The dark tank formed by pipes 702 may contain enclosed propellers 705 (illustrated in FIG. 7A) to ensure the circulation of the liquid growth medium through the cultivation system. The various systems for manipulating the growth medium may be coupled to the pipes 702. For example, nutrients dosage module 706, carbon dioxide supplying module 707, and aeration module 708 (used to strip oxygen from the culture medium) may be coupled to the pipes 702. The nutrients dosage module 706, carbon dioxide module 707, and aeration module 708 may all be controlled through a connection (not shown) to control system 710.

In the embodiment of FIG. 7B, a set of sensors 709 (for detecting pH, Temperature, Conductivity, Dissolved Oxygen, Oxidation Reduction Potential) are coupled to an inlet between the main pond 701 and the pipes 702. The sensors 709 may be coupled (not shown) to a fully automated module 710 for detection of the liquid growth medium state and control of cultivation parameters.

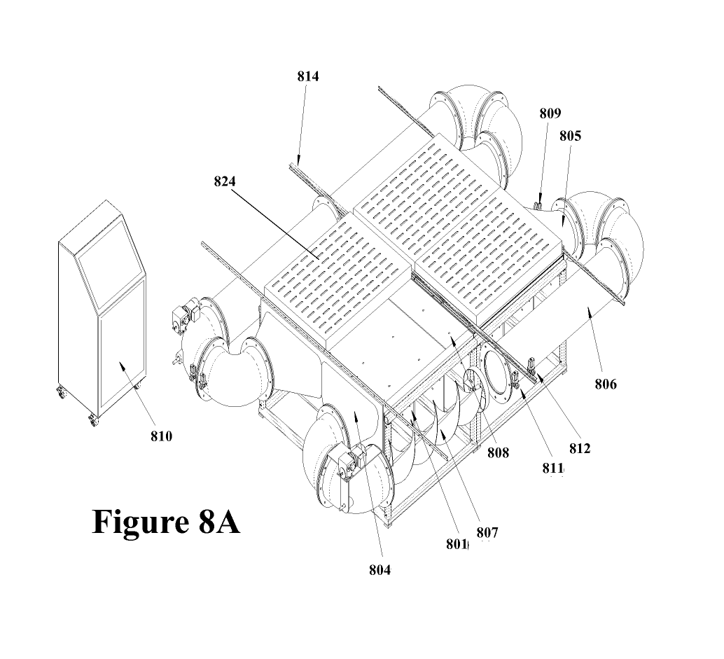

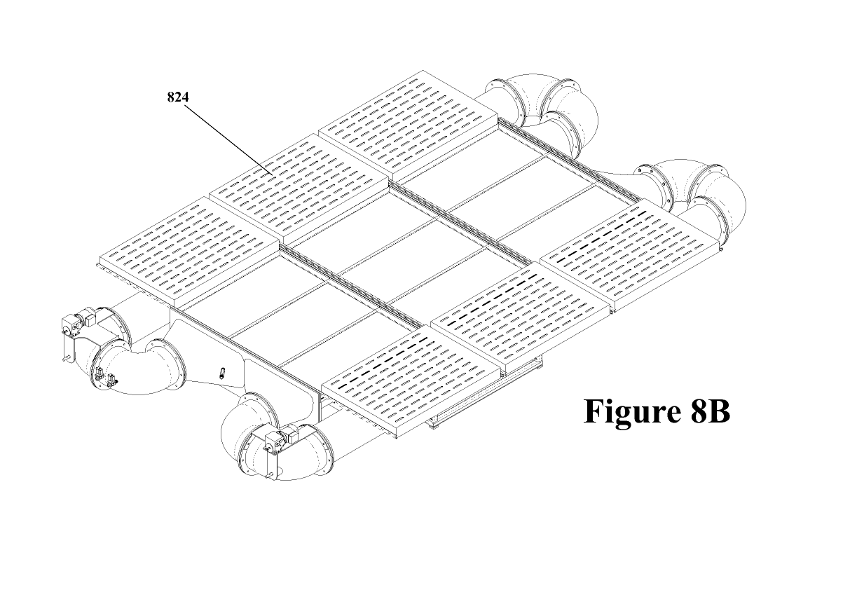

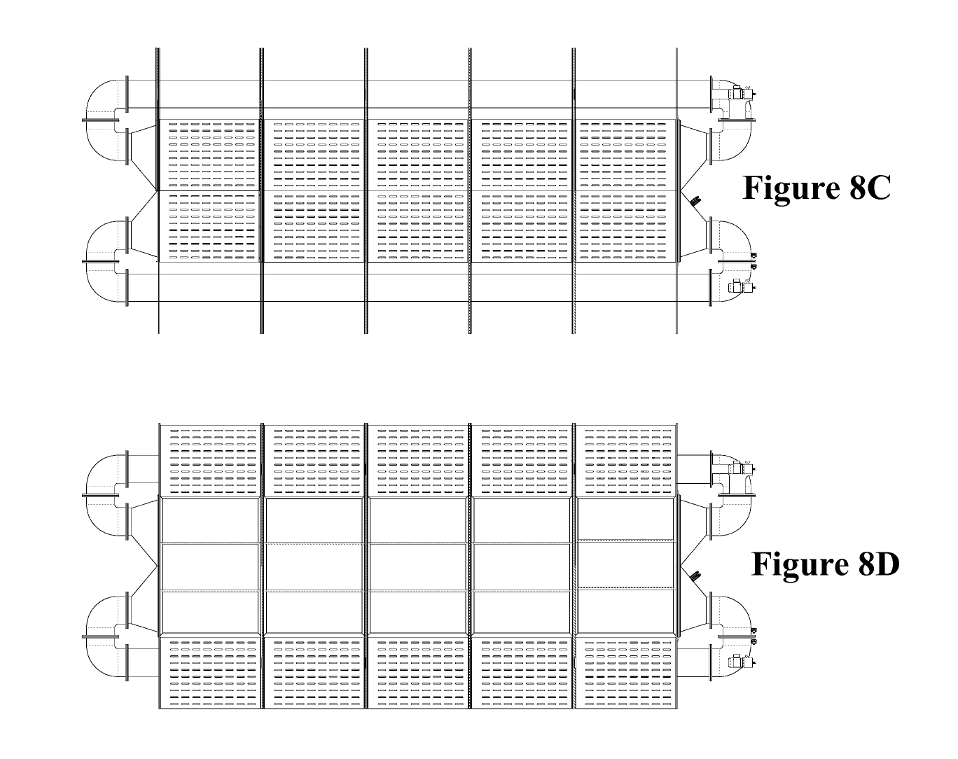

Additional details on an enclosed raceway pond are presented at the FIG. 8A. As illustrated in FIG. 8A, the enclosed raceway pond may be a modular system that is constructed from a few different prefabricated modular elements that are ended on both sides with flanges that allow assembling the modular elements to each other. For example, the raceway pond may be constructed from modular units with flanges at each end. Similarly, the dark tank may be constructed from traditional pipe segments with flanges. Sealing rings 803 may be placed between the flanges of the modular units for water-tight assembly. The number of modular elements used to construct a particular raceway pond will vary depending upon the required volume of the cultivation system being constructed. The inlet 804 and outlet 805 collectors are assembled at both ends of the enclosed raceway pond.

The inlet 804 and outlet 805 collectors are connected outside of the enclosed raceway pond to the pipes 806 that play a role of the dark tank. The diameter of pipes 806 is selected accordingly to the required volume of the dark tank. Spiral propellers 807 or other devices may be placed within the pipes 806 to ensure that the growth medium flows through the enclosed raceway pond system. The set of growth medium sensors 809 for determining the liquid growth medium state may be mounted on the inlet collector 804.

Water jets 808 for mixing the liquid growth medium in the long narrow raceway ponds may be added to the raceway pond if the mixing provided by spiral propellers is not sufficient. Mixing ensures distribution of the nutrients and gases throughout the liquid growth medium. Mixing also ensures that all of the photosynthetic organisms are able to spend some time proximate to the light source that provides energy for the photosynthetic organisms. The water jets 808 may be distributed along the raceway pond in a manner such that the next set of jets is located before the area where the turbulence activity of the previous set of jets is almost finished.

A nutrients dosing module consisting of nutrients storage tank (not shown) and dosing device with the pump 811 may be connected to the dark pipes 806. In FIG. 8A the dosing device system 811 is coupled at the inlet of spiral propellers 807 for better mixing of nutrients with the liquid growth medium. Similarly, a CO₂ supply module 812 may also be coupled at the inlet of spiral propellers 807 for better dispersing of CO₂ micro-bubbles into the growth medium. The CO₂ supply module 812 may comprise an automated CO₂ dosing device and a hollow fiber membrane diffuser. Using a porous fiber structure creates smaller bubbles and thus helps the CO₂ dissolved into the liquid growth medium.

Rails 807 may be placed on top of the raceway ponds for supporting the light panels 824. The rails 807 allow the light panels 824 to be shifted aside during daytime to expose raceway pond for sunlight (or for repair and maintenance purposes) as illustrated in FIG. 8B. Other mechanisms for removing the light panels 824 may also be used.

As previously set forth, the cultivation system is designed as a modular system that allows many different shapes and sizes of cultivation systems to be constructed. The modular design allows scaling of the closed raceway pond volume by simply adding prefabricated sections. For example, the cultivation system of FIG. 8A includes two main pond units and two pipes 806. To expand the cultivation system of FIG. 8A, a third main pond unit and pipe unit may easily be added as illustrated in FIG. 8B. FIGS. 8C and 8D illustrate a large production cultivation system built with five sets of main pond units and pipes.

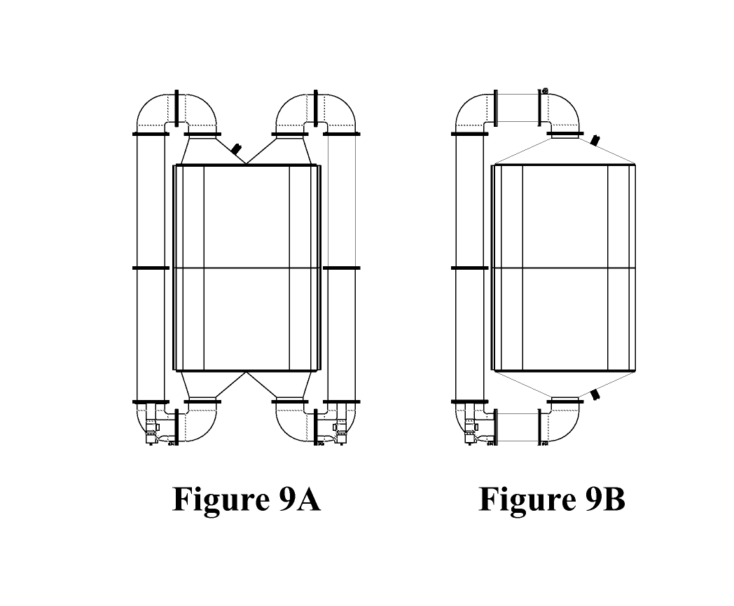

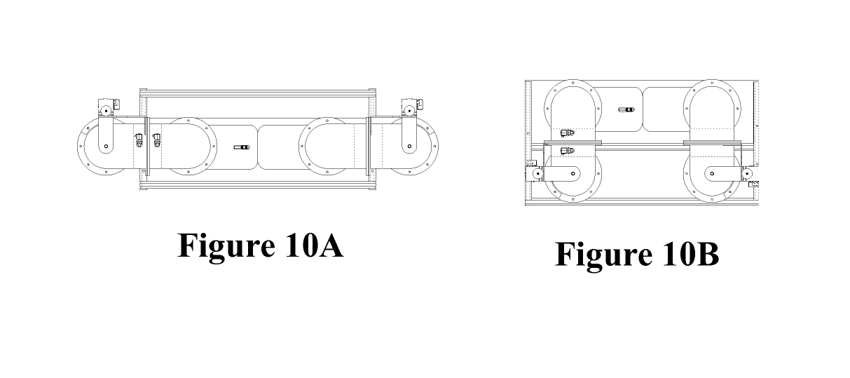

The raceway pond systems may be configured in many different manners. For example, a system may be designed with raceway pond having dark tank pipes on both sides as illustrated in FIG. 9A or with a dark tank pipe on one side only as illustrated in FIG. 9B. The dark tank pipes may have different shapes of cross-section. The raceway pond systems may be also designed with different positions of the raceway pond and the dark tank pipes. For example, the dark tank pipes may be placed horizontally adjacent to the raceway pond as illustrated in FIG. 10A or the dark tank pipes may be placed vertically underneath the raceway pond as illustrated in FIG. 10B to reduce the amount of floor space used.

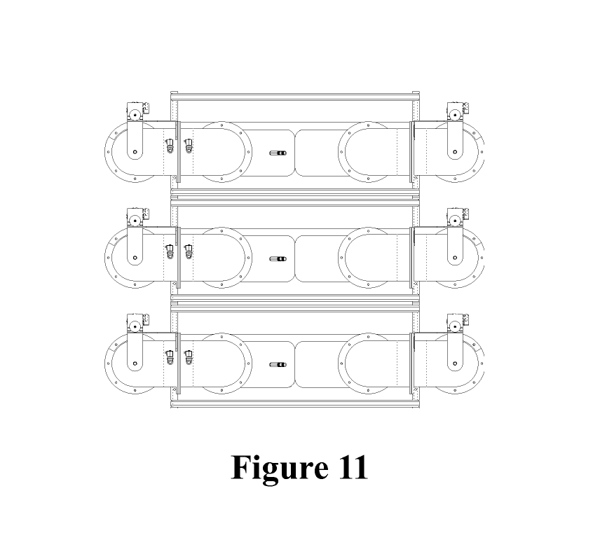

For large cultivation facilities with the limited area, the raceway ponds may be stacked one on top of another. For example, FIG. 11 illustrates three cultivation systems that have been stacked on top of each other. In this manner, a large amount of cultivation may be performed with a relatively small amount of floor space area.

The enclosed raceway ponds may be used indoors or outdoors. The enclosed raceway ponds may be used with or without artificial light sources.

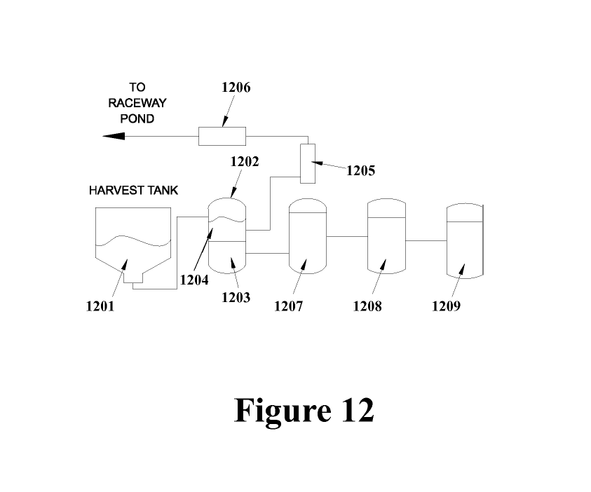

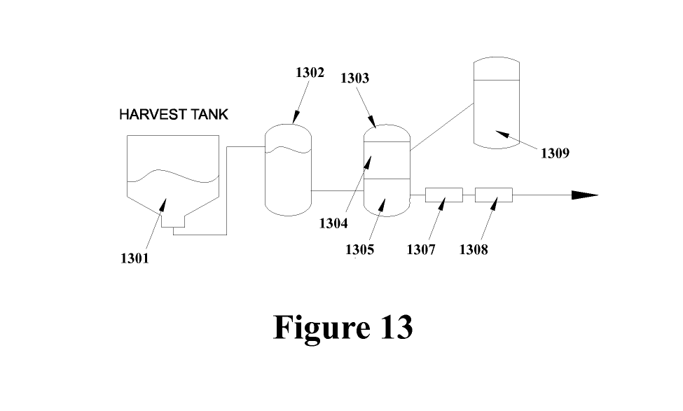

While the cell density of the photoautotrophic organisms in the liquid culture medium riches the desired level the harvesting is realized by pumping an appropriate and determined amount of liquid culture medium with photoautotrophic organisms into the harvest tank. Different process diagrams and appropriate designs may be explored for future processing of the culture medium with photoautotrophic organisms. Two examples of the harvesting process are presented at the FIG. 12 and FIG. 13.

Harvesting System

Referring back to FIG. 2, when the growth of a crop of photosynthetic organisms is complete, the liquid growth medium passes through a harvest valve 291 into a harvest tank 293 for harvest. The liquid growth medium in the harvest tank 293 is then processed with a set of biomass treatment units. FIG. 12 illustrates a first example of a harvesting system for cultivation system in greater detail.

Referring to FIG. 12, the liquid growth medium containing photosynthetic organisms to be harvested is first transferred to a harvest tank 1201. The liquid culture medium with the photosynthetic organisms passes through a filter system 1202 where the photosynthetic organism biomass 1203 is separated from the liquid culture medium 1204. The separated liquid culture medium may then be pumped through the filtration unit 1205 for purification (including removal of any remaining photosynthetic organisms and by-products of photosynthetic organisms) followed by the UV sterilization device 1206 for liquid culture medium sterilization before recycling the growth medium back into the raceway pond.

The photosynthetic organism biomass 1203 removed in the filter system 1202 is then processed by a dewatering system 1207 and drying system 1208. The dried photosynthetic organism biomass is finally processed in the appropriate processing system 1209 to obtain the final goal products from the photosynthetic organism biomass. The final goal product may be pharmaceutical source material, food supplements, or any other material collected from the harvested photosynthetic organism biomass.

If oil is the primary desired goal product from the cultivated photosynthetic organisms then a specialized system for oil harvesting may be used. FIG. 13 illustrates block diagram of a harvesting system designed for collecting oil from photosynthetic organisms. When oil is the goal product, the liquid culture medium with photosynthetic organisms can be processed without a drying stage (such as drying system 1208 illustrated in FIG. 12). Eliminating the drying stage can reduce the amount of energy consumed since drying the photosynthetic organism biomass can be a very energy intensive task.

Referring to FIG. 13, the liquid culture medium with photosynthetic organisms is moved from the harvest tank 1301 to a cell walls destruction device 1302. For energy saving purposes and to reduce the volume of liquid medium to be treated in the cell wall destruction device, a filtration device (not shown) may be used to concentrate photosynthetic organisms prior to the cell wall destruction.

The cell walls destruction device 1302 may comprise an ultrasonic destructor that uses ultrasonic waves to destroy the walls of photosynthetic organism cells. Destroying the cell walls allows the oil from the cells to be removed from the cells. Since oil is less dense than water, the oil will tend to float to the surface thus allowing for easy extraction of the oil. After processing with the cell walls destruction device 1302 the biomass is then moved into a separation unit 1303 where the oil 1304 from the cells, the remaining biomass 1305, and the liquid growth medium are separated. Many different well known oil and water separation systems can be used for this purpose. The separated oil may be collected into an oil tank 1309. The remaining biomass 1305 may be extracted and used for nutritional supplements, input biomass for another biofuel system, pharmaceuticals, or any other product that will benefit from the biomass 1305. The liquid growth medium may be passed through a filter system 1307 and a UV sterilizer 1308 before being recycled back into cultivation system.

The preceding technical disclosure is intended to be illustrative, and not restrictive. For example, the above-described embodiments (or one or more aspects thereof) may be used in combination with each other. Other embodiments will be apparent to those of skill in the art upon reviewing the above description. The scope of the claims should, therefore, be determined with reference to the appended claims, along with the full scope of equivalents to which such claims are entitled. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim is still deemed to fall within the scope of that claim. Moreover, in the following claims, the terms “first,” “second,” and “third,” etc. are used merely as labels, and are not intended to impose numerical requirements on their objects.

The Abstract is provided to comply with 37 C.F.R. §1.72(b), which requires that it allow the reader to quickly ascertain the nature of the technical disclosure. The abstract is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment.

We claim:

1. A replaceable photobioreactor for use within a cultivation system for growing photosynthetic organisms in a liquid growth medium, said replaceable photobioreactor comprising:
a first transparent plastic sheet;
a second plastic sheet, said second plastic sheet welded to said first transparent plastic sheet in a manner that creates a continuous cross-flow channel;
a first connecting pipe at a first end of said continuous cross-flow channel; and
a second connecting pipe at a second end of said continuous cross-flow channel.

2. The replaceable photobioreactor as set forth in claim 1 wherein said second plastic sheet is also a transparent plastic sheet.

3. The replaceable photobioreactor as set forth in claim 1 wherein said second plastic sheet has a reflective surface facing said first transparent plastic sheet.

4. The replaceable photobioreactor as set forth in claim 1, said photobioreactor further comprising:
hanging system connector for hanging said replaceable photobioreactor.

5. The replaceable photobioreactor as set forth in claim 4 wherein said hanging system connector comprises a plurality of small holes through said first transparent plastic sheet and said second plastic sheet.

6. The replaceable photobioreactor as set forth in claim 1 wherein said continuous cross-flow channel comprises a series of connected horizontal flow channels.

7. The replaceable photobioreactor as set forth in claim 6 wherein said connected horizontal flow channels have a half-circular cross section.

8. The replaceable photobioreactor as set forth in claim 6 wherein said connected horizontal flow channels have a circular cross section.

9. The replaceable photobioreactor as set forth in claim 6 wherein said connected horizontal flow channels have a square cross section.

10. The replaceable photobioreactor as set forth in claim 6 wherein said connected horizontal flow channels are formed by a top weld and a bottom weld in an alternating pattern.

11. A method of creating a replaceable photobioreactor for use within a cultivation system for growing photosynthetic organisms in a liquid growth medium, said method comprising:
laying a first transparent plastic sheet on top of a second transparent plastic sheet;
welding said second plastic sheet to said first transparent plastic sheet in a manner that creates a continuous cross-flow channel;
creating a first opening at a first end of said continuous cross-flow channel; and
creating a second opening at a second end of said continuous cross-flow channel.

12. The method of creating a replaceable photobioreactor as set forth in claim 11 wherein said second plastic sheet is also a transparent plastic sheet.

13. The method of creating a replaceable photobioreactor as set forth in claim 11 wherein said second plastic sheet has a reflective surface facing said first transparent plastic sheet.

14. The method of creating a replaceable photobioreactor as set forth in claim 11, said method further comprising:
creating a hanging system connector on a first end of said replaceable photobioreactor for hanging said replaceable photobioreactor.

15. The method of creating a replaceable photobioreactor as set forth in claim 14 wherein said hanging system connector comprises a plurality of small holes through said first transparent plastic sheet and said second plastic sheet.

16. The method of creating a replaceable photobioreactor as set forth in claim 11 wherein said continuous cross-flow channel comprises a series of connected horizontal flow channels.

17. The method of creating a replaceable photobioreactor as set forth in claim 6 wherein said connected horizontal flow channels have a half-circular cross section.

18. The method of creating a replaceable photobioreactor as set forth in claim 16 wherein said connected horizontal flow channels have a circular cross section.

19. The method of creating a replaceable photobioreactor as set forth in claim 16 wherein said connected horizontal flow channels have a square cross section.

20. The method of creating a replaceable photobioreactor as set forth in claim 16 wherein said connected horizontal flow channels are formed by a top weld and a bottom weld in an alternating pattern.
